(19) United States
(12) Patent Application Publication
(10) Pub. No.: US 2008/0051722 A1
(43) Pub. Date: Feb. 28, 2008

(54) Methods and apparatus for determining the location of implanted ports

(75) Inventors: James Christopher David Ellsmere, Brookline, MA (US); William Mercer Wells III, Cambridge, MA (US)

Correspondence Address: WOLF GREENFIELD & SACKS, P.C., 600 ATLANTIC AVENUE, BOSTON, MA, 02210-2206, US

(73) Assignee: Beth Israel Deaconess Medical Center, Inc., Boston, MA (US)

(21) Appl. No.: 11/811,713

(22) Filed: Jun. 12, 2007

Related U.S. Application Data
(60) Provisional application No. 60/812,886, filed on Jun. 12, 2006.

(51) Int. Cl. A61M 39/02

(52) U.S. Cl. 604/175

(57) ABSTRACT

In one aspect, a device for locating a subcutaneously implanted port is provided. The device includes a port beacon adapted to be implanted proximate the subcutaneously implanted port, the port beacon configured to generate a beacon signal, and a port locator including at least one first coil responsive to the beacon signal, the at least one first coil configured to generate a locator signal in response to the beacon signal, the port locator further includes a controller to receive the locator signal and generate at least one proximity measurement indicative of a location of the port beacon based, at least in part, on the locator signal.

### RELATED APPLICATION

This application claims priority under 35 U.S.C. § 119(e) to U.S. Provisional Application Ser. No. 60/812,886, entitled “METHODS AND APPARATUS FOR DETERMINING THE LOCATION OF IMPLANTED PORTS,” filed on Jun. 12, 2006, which is herein incorporated by reference in its entirety.

### FIELD OF THE INVENTION

The present invention relates to methods and apparatus for locating a subcutaneously implanted access port, and more particularly, to generally office-based methods and apparatus for the same.

### BACKGROUND

Ports implanted below the skin provide access to a variety of subcutaneously implanted medical devices. For example, implanted ports allow access to veins, laparoscopic adjustable gastric banding (LABG), soft tissue expanders, analgesic pumps, etc. The number of implanted ports has been increasing rapidly, in part, to support the recent spike in LABG procedures. Recent statistics have shown that more than 8 million people in the United States have a body mass index (BMI) of 40 or higher, and an additional 23 million have a BMI between 35 and 40. Many of these individuals have significant obesity related co-morbidities such as hypertension, type II diabetes, hyperlipidemia, obstructive sleep apnea, asthma, gastro-esophageal reflux disease and arthritis. Weight loss surgery (e.g., LABG) remains the most effective treatment for morbid obesity and the co-morbidities associated with morbid obesity.

Accessing ports that have been implanted below the skin is a daily event in hospitals around the world. As discussed above, ports are used for a range of applications, including, but not limited to, long term venous access, soft tissue expanders, analgesic pumps and adjustable gastric bands. Ideally, port access can be performed in the office or at the bedside of the patient by palpating the port and then inserting a needle into it. FIG. 1 illustrates an exemplary palpated port and a needle positioned to be inserted into the port to, for example, access a corresponding vein, insert material to expand tissue, adjust the amount of water in a gastric band, etc. It should be appreciated that the port is implanted under the skin such that, absent means to locate the port, personnel attempting to insert the needle would be blind to the location of the implanted port.

For example, in a LAGB procedure a gastric band 200 (e.g., the gastric band illustrated in FIG. 2) is connected to a port 210 which may be sutured to abdominal wall musculature below the subcutaneous fat of a patient undergoing the procedure. The ports may be accessed with a non-coring needle in follow-up visits to adjust the amount of water in the band and effectively adjust the tightness around the stomach by palpitating the port. If the port is not palpable, or it remains difficult to access the port after palpitation, physicians may use imaging to assisting in guiding the needle into the port. That is, the patient may be imaged using, for example, x-ray technology to locate the port such that the needle can be guided into the port.

### SUMMARY OF THE INVENTION

Some embodiments according to the present invention include a port beacon adapted to be implanted with a subcutaneous port to facilitate locating the implanted port, the port beacon comprising at least one coil configured to obtain first energy from an externally provided excitation signal and to transmit an echo signal in response to the excitation signal, and a transmit and receive circuit coupled to the at least one coil, the transmit and receive circuit configured to receive the first energy obtained by the at least one coil and provide the at least one coil with second energy derived, at least in part, from the first energy to cause the at least one coil to transmit the echo signal.

Some embodiments according to the present invention include a port locator for facilitating location of an port implanted subcutaneously, the port locator comprising at least one coil responsive to an electromagnetic beacon signal generated at the port, the at least one coil configured to generate a locator signal in response to the electromagnetic beacon signal, and a controller configure to receive the locator signal and generate at least one proximity measurement indicative of a location of the port based, at least in part, on the locator signal.

Some embodiments according to the present invention include a device for locating a subcutaneously implanted port, the device comprising a port beacon adapted to be implanted proximate the subcutaneously implanted port, the port beacon configured to generate a beacon signal, and a port locator comprising at least one first coil responsive to the beacon signal, the at least one first coil configured to generate a locator signal in response to the beacon signal, the port locator further comprising a controller to receive the locator signal and generate at least one proximity measurement indicative of a location of the port beacon based, at least in part, on the locator signal.

Some embodiments according to the present invention include a method of locating a port beacon implanted subcutaneously in a patient proximate an implanted port, the method comprising generating, at the port beacon, an electromagnetic beacon signal, detecting the electromagnetic beacon signal via at least one first coil provided external to the patient, and determining at least one proximity measurement indicative of a location of the port beacon based, at least in part, on the detected electromagnetic beacon signal.

### BRIEF DESCRIPTION OF THE DRAWINGS

FIG. 1 illustrates an exemplary port showing a needle accessing the port;

FIG. 2 illustrates a gastric band attached to a port for implanting in a patient undergoing a laparoscopic adjustable gastric banding (LABG) procedure;

FIG. 3 illustrates a port locator system for locating an implanted port, in accordance with some embodiments of the present invention;

FIG. 4 illustrates an implanted port and port beacon for transmitting a detectable signal, in accordance with some embodiments of the present invention;

FIG. 5A illustrates a block diagram of a port locator using a single coil for facilitating the location of an implanted port, in accordance with some embodiments of the present invention;

FIG. 5B illustrates a block diagram of a port locator using multiple coils for facilitating the location of an implanted port, in accordance with some embodiments of the present invention;

FIG. 6 illustrates a method for locating an implanted port using a port locator and an implanted port beacon, in accordance with some embodiments of the present invention;

FIGS. 7A and 7B illustrate a coil model for determining location and orientation information from a plurality of measurements obtained via a multiple coil configuration, in accordance with some embodiments of the present invention.

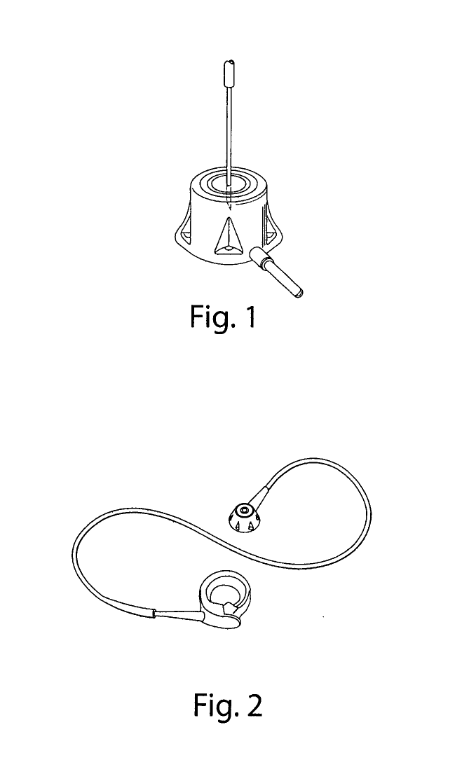

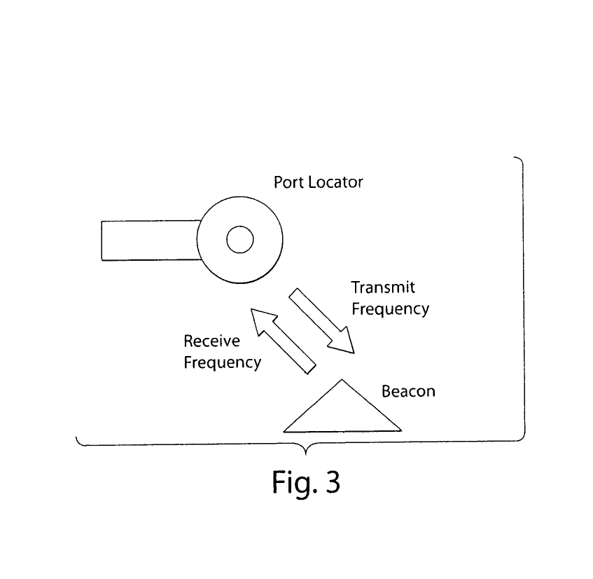

### DETAILED DESCRIPTION

As discussed above, implanted ports are often accessed by palpating the port. When the port is not palpable and/or palpitation is ineffective to make the port accessible by a needle, imaging of the patient may be used to locate the port and guide the needle into the port. A standard imaging technique for facilitate port access is the use of fluoroscopy. The radio-opaque ports and the needle can be easily seen with real-time x-ray imaging. In particular, the position and orientation of the port can be seen with enough detail to accurately guide the needle (which also can be viewed in real time) into the access port. The technique for directing the needle into the port using x-ray imaging is relatively simple to learn.

However, the imaging procedure is not office-based. In particular, image guided port access requires either an unscheduled procedure in the radiology department or scheduling an additional visit. Either scenario is problematic for both physicians and patients. Another disadvantage of imaging is radiation exposure. Physicians who are repeatedly exposed need to gown with lead to limit exposure to their neck and torso. In addition, there is considerable cost in purchasing, storing and maintaining imaging equipment.

In certain limited cases, ultrasonography can be used as an alternative to fluoroscopy. The main problem with ultrasonography is that it is not intuitive. Developing the skill to use ultrasound requires undergoing a lengthy training program and is relatively complicated. Maintaining the skills necessary to perform ultrasononography guided port access also requires regular practice of the technique, such that proper qualified physicians/technicians may not be readily available. Ultrasound guidance is particularly difficult for lesions that are superficial (<1 cm below the skin) or deep (>5 cm below the skin). There are also considerable costs in purchasing, storing and maintaining ultrasound equipment.

Some conventional approaches to solving problems associated with locating implanted ports include implanting one or more magnets under the skin in association with the implanted port. An external instrument is constructed having one or more magnets that align in a predetermined fashion when located proximate the implanted port. That is, the implanted magnets interact with magnets on a locating device to indicate alignment. However, implanting magnets has the drawback of preventing subsequent magnetic resonance imaging (MRI) procedures. In particular, an implanted magnet may be dangerous when placed within the high magnetic field strengths typically associated with MRI. In addition, solutions using magnets may not provide sufficient information to determine the correct position (much less orientation) of the implanted port.

Other conventional approaches to implanted port detection include implanting a circuit including one or more light emitting diodes (LEDs) proximate the port. When an external device is passed over the port to energize the circuit, the LEDs light up to indicate where the port is located. Although visible or infra red light does penetrate soft tissue, light may be substantially scattered in the process, which may render accurate location detection difficult. As a result, this solution may only work adequately for very shallow implantation depths, and may not be appropriate for some port implantations and/or certain patients (e.g., patients who are overweight, which patients undergoing LABG procedures, for example, generally are). Accordingly, the various conventional techniques that attempt to locate implanted ports without the use of x-ray imaging, ultrasonography or other relatively complex out of office procedures have drawbacks to the extent that none of these office-based techniques have garnered acceptance by the medical community.

Applicant has appreciated that an office-based device for locating implanted ports without one or more of the drawbacks described above would allow port location to be handled relatively efficiently without having to set up a separate appointment, for example, with a radiologist and/or obviate the need to perform relatively complex procedures to locate an implanted port. Applicant has appreciated that one or more of the above shortcomings may be overcome by providing a port having location means that can be detected by an external device. In one embodiment, the port is implanted with a port beacon adapted to emit a beacon signal that may be detected external to the patient in which the port has been implanted. A port locator device is adapted to detect the beacon signal to facilitate location of the port. Once the port has been located, the port locator device may be used to guide a needle to the implanted port.

Following below are more detailed descriptions of various concepts related to, and embodiments of, methods and apparatus according to the present invention. It should be appreciated that various aspects of the invention described herein may be implemented in any of numerous ways. Examples of specific implementations are provided herein for illustrative purposes only. In addition, the various aspects of the invention described in the embodiments below may be used alone or in any combination, and are not limited to the combinations explicitly described herein.

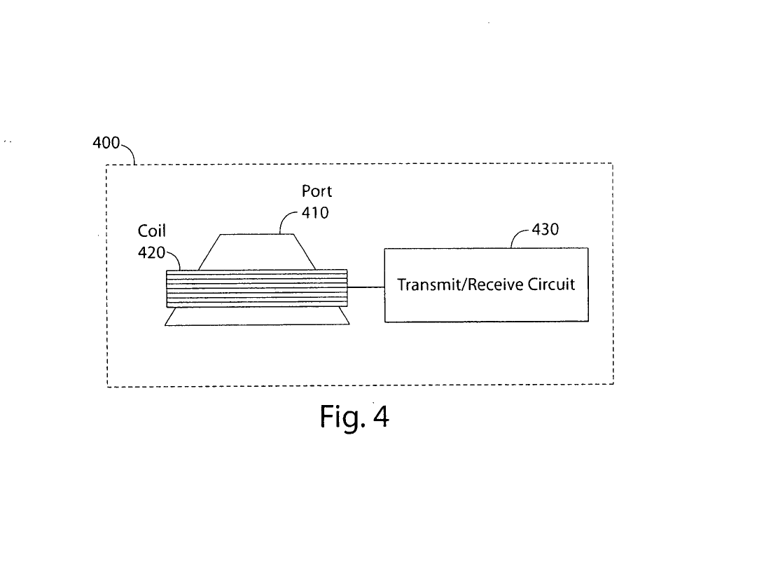

FIG. 3 illustrates a system for locating an implanted port, in accordance with some embodiments of the present invention. The system includes a device, referred to as a beacon, that is attached or fastened to the port at the time of implantation. The beacon may be an active or passive device. The term “passive” refers herein to a device that does not maintain its own independent power source, but instead may generate power based on one or more external sources of energy, or forces from some other means. For example, an induction coil is one type of passive device that may generate current based on externally supplied electromagnetic energy. Other passive devices include piezoelectric devices, solar cells, thermal converters, etc. A passive beacon may not need replacement of a power source (as would be the case in active devices), making implementations using passive devices preferable. However, active devices (e.g., a battery powered device) may be used as well, as the aspects of the invention are not limited in this respect.

The system illustrated in FIG. 3 also includes a port locator adapted to locate the beacon. In one embodiment, the beacon is a passive device that is attached to the port at the time of implantation. The beacon may be activated by the port locator, wherein the beacon emits a signal in response to the port locator that is indicative of the location of the port. The signal may then be used by the port locator to determine how closely the port locator is aligned with the implanted port, where the port is located and/or the optimal angle at which to approach it. It should be appreciated that the beacon may be an active device, as the aspects of the invention are not limited in this respect. However, as a general matter, active devices require additional power to operate, which may complicate the device and/or impact how long the beacon can operate independently without a recharge.

The port locator may be a small battery powered hand held device that also has one or several antenna coils, a display, speaker, and a bore that serves as a needle guide. Once activated, the port locator transmits a signal to the beacon. In embodiments using a passive beacon, the beacon may absorb power from the signal (referred to as an excitation signal) generated by the port locator (e.g., via induction) and in turn transmits a response signal detectable by the port locator (e.g., via one or more port locator coils). A beacon coil may be arranged so that the axis of the coil corresponds to the preferred perpendicular needle approach to the center of the port. The port locator receiving coil may be arranged so that its axis corresponds to the bore of the port locator needle guide; and the strength of the signal that is received from the beacon will be maximized when the coils are close to each other and oriented along the same axis, which corresponds to the desired needle orientation. That is, the signal may be maximal when the needle, via the port locator bore, is perpendicular to the port and aimed at its center.

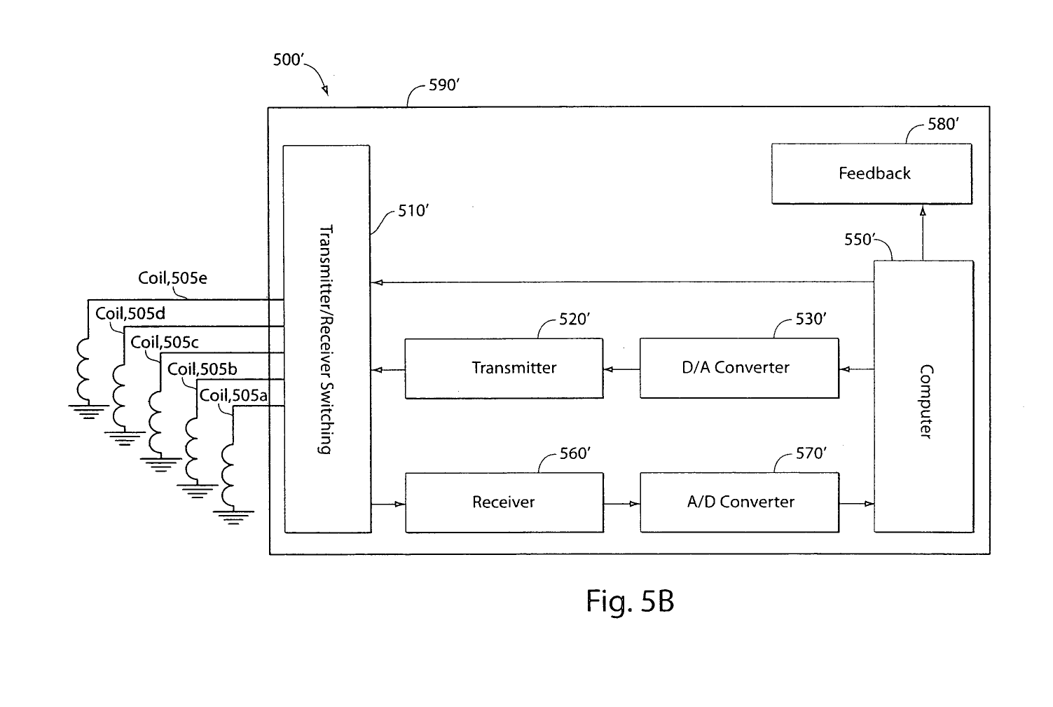

FIG. 5 illustrates the magnetic part of the beacon's transmitted field near the beacon, which is symmetric about the axis of the coil, and may contain proximity and/or orientation information. It should be appreciated that any type of electromagnetic signal may be used to locate the port, as the aspects of the present invention are not limited for use with any particular type of signal. Any other beacon/locator types and configurations may used to locate a port implanted within a patient, as the aspects of the invention are not limited in this respect. Any of various coils in combination with a capacitor may be used as the beacon and a tunable oscillator may be used as the port locator. However, the aspects of the invention are not limited for used with any particular implementation, circuit components, etc.

In some embodiments, the feedback from the port locator to the physician performing the port access may be both visual and audio. The visual feedback may show the user the direction and orientation to move the needle. The audio signal may vary in relation to the strength of the received signal, so that as the needle moves closer to the port and the optimal trajectory, the audio tone will have a higher frequency or higher volume. Video and audio feedback may be used alone or in combination with another, and other feedback methods may be implemented to assist an operator in accurately locating the port and optimally positioning the needle for access, as the aspects of the invention are not limited in this respect.

It should be appreciated that the above described port locator system is relatively small and portable, facilitating its use during routine office visits. In addition, limited or no special training is required to use the device, obviating the need to have a specialized and highly trained operator available to perform port access. In addition, the port locator system is generally safe both for the patient and the operator of the device. In particular, dangerous high energy radiation used in conventional imaging techniques may not be necessary to locate a port accurately.

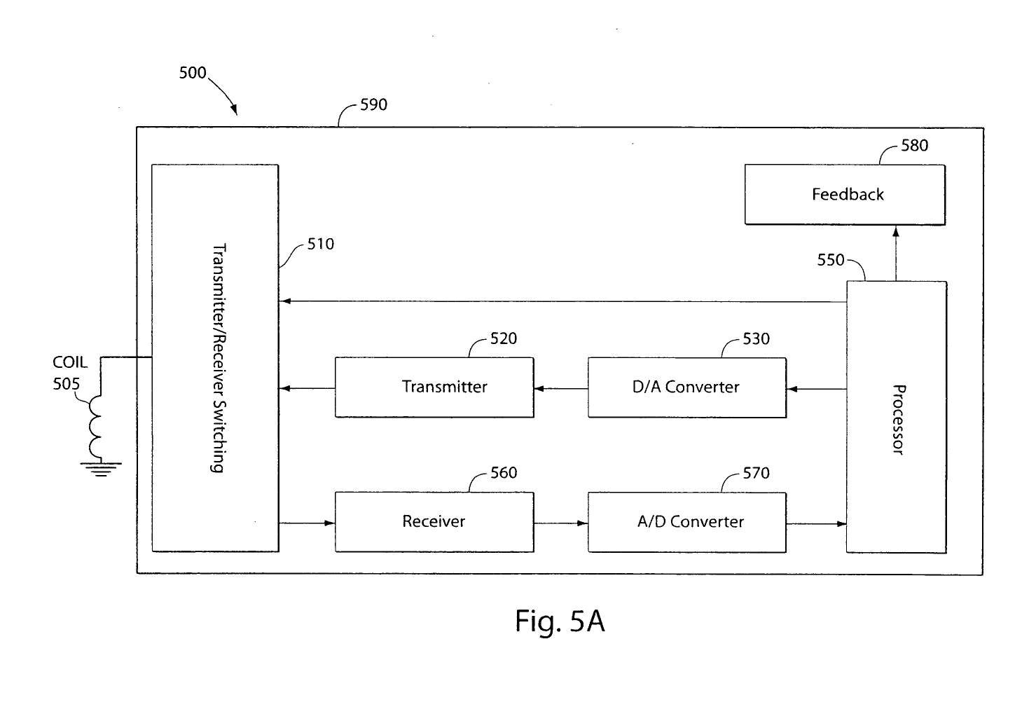

FIG. 4 illustrates a port beacon to facilitate locating an implanted port, in accordance with some embodiments of the present invention. Implant 400 includes a port 410 implanted subcutaneously, an induction coil 420, and transmit/receive circuit 430. Induction coil 420 may be attached to the coil or implanted proximate the coil in a generally known location relative to port 410. Preferably, induction coil 420 is attached or affixed to the port at the time of implantation such that signals indicative of the location of the port beacon are also indicative of the location of the port. For example, as shown in FIG. 4, the coil may be attached to the port so that the axis of the coil corresponds to the preferred perpendicular needle approach to the center or opening of the port. However, the coil may be attached to or arranged proximate the port in other ways, as the aspects of the invention are not limited in this respect.

The induction coil may be coupled to transmit/receive circuit 430 to both receive signals provided externally and to generate signals to be transmitted. Preferably the transmit/receive electronics form a passive circuit (i.e., it does not maintain an independent power source) so that a power source does not have to be replaced on a periodic basis. However, transmit/receive electronics may be an active circuit as well, for example, a battery powered circuit. In some embodiments, the transmit/receive electronics include a passive circuit having a capacitor for storing energy obtained from an externally provided signal, and an oscillator for driving the induction coil at a desired frequency. For example, the port beacon may include a 27 mm coil of 5 mH coupled to a passive electronic circuit having a resonating capacitor, a rectifier, a 10 μF storage capacitor, and a transistor oscillator circuit that uses the induction coil and resonating capacitor as a tank circuit. However, any circuit components and configuration may be used, as the aspects of the invention are not limited in this respect.

As discussed above, the terms active and passive are used herein to describe the presence and absence of an independent power source, respectively, and not as traditionally used in describing circuit elements. For example, the capacitor in a passive circuit stores energy received external to the circuit (e.g., external the implant) and does not have its own independent power source. The passive circuit may operate by storing energy supplied externally and use the stored energy to emit a detectable signal. The term “external” is used herein to describe signals, energy, etc. provided by means that are not part of the port or port beacon apparatus.

When electromagnetic energy is applied externally (e.g., transmitted via the port locator as discussed in further detail below), induced current in the implanted coil may be used to charge the capacitor. Subsequently, the energy stored in the capacitor may be used to drive the oscillator which in turn delivers power to the induction coil. The excited induction coil transmits an electromagnetic beacon signal that can be detected externally to assist in locating the implanted port. The term “beacon signal” refers to any signal provided by the port beacon adapted to be detectable external to the port beacon. A beacon signal may be referred to as an echo signal when generated in response to an excitation signal. The passive circuit may include other components to facilitate obtaining power from an external source and using the power to drive the implanted induction coil. For example, the passive circuit may include a rectifier to rectify the induced current in order to charge the capacitor. In addition, one or more switches may be used to switch between the charge and discharge stage (e.g., a receive and transmit mode, respectively).

It should be appreciated that, in some embodiments, an implanted coil operates as both a receiver (e.g., the implanted coil is responsive to an excitation pulse generated by the port locator) and a transmitter (e.g., the implanted coil transmits the echo signal). However, other embodiments may use different components to achieve reception and transmission. The circuit described is merely exemplary and any circuit capable of providing a detectable signal may used, as the aspects of the invention are not limited in this respect.

FIG. 5A illustrates a port locator for determining the location of an implanted port, in accordance with some embodiments of the present invention. Port locator 500 may be adapted to operate in conjunction with the port beacon illustrated in FIG. 4 and described in the foregoing. The port locator includes a coil 505 adapted to both transmit and detect electromagnetic signals, and a controller 590 to drive coil 505, and to receive and process signals generated by the coil. Controller 590 may include any number, type and configuration of components, some exemplary embodiments of which are discussed below. For example, controller 590 may include transmitter/receiver switching circuit that operates both to drive coil 505 when in a transmit mode and receive signals detected by coil 505 when in a receive mode. Controller 590 also may include a processor 550 capable of synthesizing desired transmit signals and processing received signals to determine a location of an implanted port.

In particular, processor 550 may include a memory capable of storing one or more programs that generate a digital signal representative of an excitation signal desired to be transmitted to the implant via coil 505. A digital-to-analog (D/A) converter 530 may be provided to convert the digital signals into an analog signal which transmitter 520 uses to drive coil 505. When the port locator is in a transmit mode, transmitter/receive switching 510 passes the drive signal from transmitter 520 to the coil, which generates an electromagnetic signal (e.g., an excitation signal) in response to the drive signal. Conversely, when in a receive mode, transmitter/receive switching passes electromagnetic signals detected by coil 505 to receiver 560, which generates analog signals in response to the electromagnetic signals received at coil 505. The analog signals from the receiver may be converted to digital signals by analog-to-digital (A/D) converter 570. Processor 550 may then process the digital signal received from the A/D converter to facilitate locating the implanted port.

Processor 550 may execute a program designed to analyze the detected signals and determine how closely aligned the port locator is with the implanted port. For example, a magnitude of the detected signal may indicate how close or how far the port locator is from optimal alignment. That is, when the port locator is optimally aligned with the implanted port, maximal signal strengths may be detected by coil 505. Processor 550 may generate a human-comprehensible output to indicate to a user (e.g., a doctor or nurse attempting to locate the port) how closely the port locator is aligned with the implanted port. For example, port locator 500 may include feedback 580 capable of presenting feedback to a user indicative of the spatial relationship and/or alignment between the port locator and the implanted port.

In some embodiments, feedback 580 includes either audio feedback, visual feedback or both. For example, feedback 580 may include LEDs to indicate proximity to the implanted port. For example, a plurality of LEDs may be provided, wherein the number of LEDs that are energized is related to the strength of the signal detected from the implanted port. The user may move the port locator around until all of the LEDs are energized. Different colored LEDs may also be used to indicate proximity to optimal alignment with the implanted port. In some embodiments, audio signals are generated to guide the user into alignment with the implanted port. For example, the frequency of a generated tone may vary in accordance with the strength of the signal detected from the implanted port. In some embodiments, both audio and visual cues are provided to assist a user in locating the implanted port. The above described audio and/or visual feedback mechanisms are merely exemplary and any feedback techniques may be used, as the aspects of the invention are not limited in this respect.

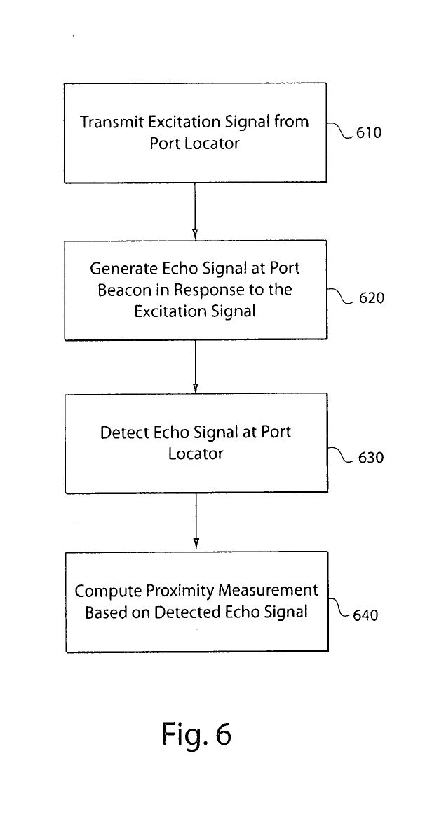

FIG. 6 is a flowchart illustrating a method of locating an implanted port, in accordance with some embodiments of the present invention. Method 600 may be used in connection with a port implanted with a port beacon and an external port locator adapted to detect signals generated by the implanted port beacon (e.g., any of the port beacons and port locators described herein). In act 610, an excitation signal is generated and transmitted by a port locator. In some embodiments, the excitation signal is an electromagnetic signal generated via a coil. For example, the port locator may be similar to port locator 500 and transmitter 520 may drive coil 505 to generate a suitable excitation signal.

In act 620, the port beacon generates an echo signal in response to the excitation signal generated by the port locator. For example, the port beacon may be similar to the port beacon illustrated in FIG. 4 and the excitation signal may induce a current in induction coil 420. The induced current may be stored by transmit/receive circuit 430. The stored energy may then be used to drive induction coil 420 to generate the echo signal. In act 630, the port locator detects the echo signal generated by the port beacon. For example, in embodiments employing a port locator similar to port locator 500, coil 505 may be used to detect the echo signal. Coil 505 may produce a locator signal (e.g., generate a current) proportional to the signal strength of the echo signal. A “locator signal” refers herein to any signal generated by a coil in response to a beacon signal, or derived from a signal generated by a coil in response to a beacon signal.

In act 640, one or more proximity measurements may be computed based, at least in part, on the locator signal generated by the coil (act 640). For example, a signal indicative of a magnitude of the locator signal may be provided to a processor to compute one or more values representative of how closely the port locator is aligned with the implanted port and/or to compute position or orientation information related to the position and orientation of the implanted port (as discussed in further detail below). The one or more proximity measurements may be indicated to a user via feedback, e.g., an audio, visual and/or numerical feedback, or other feedback mechanism adapted to assist the user in locating an implanted port.

It should be appreciated that as the port locator is moved around on a surface of a patient in an area under which the implanted port is expected to be located, method 600 may be repeated at each new location on the surface to obtain indication of how closely the port locator is aligned with the implanted port. The user (e.g., a doctor or nurse) may monitor the feedback from the port locator to determine the most likely location of the implanted port (e.g., the location at which the detected echo signal strength is greatest, as indicated by the feedback mechanism of the port locator). In this manner, a relatively simple to use, office-based device for locating implanted ports may be achieved.

The method described above in connection with FIG. 6 may be implemented in numerous ways, including employing frequency domain multiplexing (FDM) techniques and time domain multiplexing (TDM) techniques, examples of which are described below. FDM and TDM techniques differ generally by how the excitation signal and echo signal are distinguished (i.e., using different frequencies and time slices, respectively). In one embodiment using FDM techniques, the excitation signal is transmitted continuously at a first frequency (e.g., 40 kHz). The electronics of the port beacon may include a rectifier-based harmonic generator that, in response to the excitation signal, generates an echo signal having third and higher harmonics of the first frequency. Accordingly, the excitation and echoes signals may be distinguished in the frequency domain.

In one embodiment employing TDM techniques, the excitation signal is transmitted in pulses. For example, the port locator may generate a 40 kHz pulse of duration 250 ms to be transmitted by a port locator coil. The port beacon then uses the excitation pulse to generate an echo signal. For example, a port beacon coil may generate an induced current as a result of the excitation pulse. The induced current may be rectified and used to charge an energy storage capacitor. After the excitation pulse is complete, the port locator (e.g., the transmit/receive switching) may switch to receive mode. The storage capacitor releases its stored energy to energize a low power oscillator (e.g., a 40 kHz oscillator) that drives the implant coil to transmit an echo signal. A port locator coil may then detect the echo signal which may then be processed to determine a proximity measurement of the implanted port (e.g., as discussed above in connection with FIGS. 4 and 5A). It should be appreciated that the excitation pulse may be of any desired frequency or combination of frequencies and may be pulsed for any desired duration using any desired duty cycle, as the aspects of the invention are not limited for use with any particular type of excitation signal.

It should be appreciated that the above techniques are merely exemplary and numerous other techniques and variations on the above techniques may be used, as the aspects of the invention are not limited in this respect. In some embodiments, coils are used as both transmitters and receivers at both the port locator and port beacon. For example, one or more port locator coils may be used to transmit the excitation signal and one or more of the same coils may be used to detect an echo signal generated by the port beacon. Similarly, one or more port beacon coils may receive the excitation signal and one or more of the same coils may be used to transmit the echo signal. However, coils need not be used as both transmitters and receivers, as the aspects of the invention are not limited in this respect.

As discussed above, determining the location of an implanted port facilitates accessing the port, typically via a needle, syringe or other device capable of penetrating the skin and entering an opening or septum in the implanted port. The opening in the port may be self-sealing or include other mechanisms to allow a needle to penetrate the port, while preventing liquid, air or other subject matter from exiting the port. Inaccurate detection of port location may lead to accidental puncturing of the port by the needle, or otherwise compromising the integrity of the port. Even in instances where inaccurate port location does not result in damaging the port, multiple attempts to access the port may be uncomfortable or painful for the patient.

Applicant has recognized that single measurements obtained from a port beacon may, in some circumstances, be insufficient to locate the port with suitable precision. For example, movement by the patient may cause the implanted port to shift and rotate within the body such that the axis of the port opening is not orthogonal with the surface of the skin. As a result, it may be desirable to obtain information indicative of both position and orientation of the implanted port with respect to the port locator. Applicant has appreciated that a port locator capable of obtaining multiple measurements at a given location may facilitate acquisition of both position and orientation information with respect to the location of an implanted port.

FIG. 5B illustrates a port locator adapted to obtain multiple measurements from an implanted port beacon at a given location of the port locator, in accordance with some embodiments of the present invention. Port locator 500′ may be similar to the port locator illustrated in FIG. 5A. However, port locator 500′ includes multiple coils (e.g., coils 505a-505e) and controller 590′ may include transmit/receive switching 510′ adapted to, in addition to performing switching between transmit and receive modes, select amongst the multiple coils as to which coil to operate in the transmit and receive modes. That is, transmit/receive switching may sequence transmitting and receiving at different combinations of coils to obtain a plurality of measurements at a given location of the port locator. While port locator 500′ includes five coils, any number of coils may be used to obtain the plurality of measurements, as the aspects of the invention are not limited in this respect.

Processor 550′ may be adapted to generate the appropriate drive sequences for the various coil combinations, and to perform an analysis based on a plurality of measurements (as opposed to the single measurement per location obtained using the port locator device in FIG. 5A). In particular, processor 550′ may be configured to determine both position and orientation information from the plurality of measurements, as discussed in further detail below. The feedback mechanism may include indicators of both position and orientation proximity, or may include a single indicator of proximity based on both position and orientation information. As discussed above, the feedback mechanism may include audio, visual or other feedback means, or any combination thereof, as the aspects of the invention are not limited in this respect.

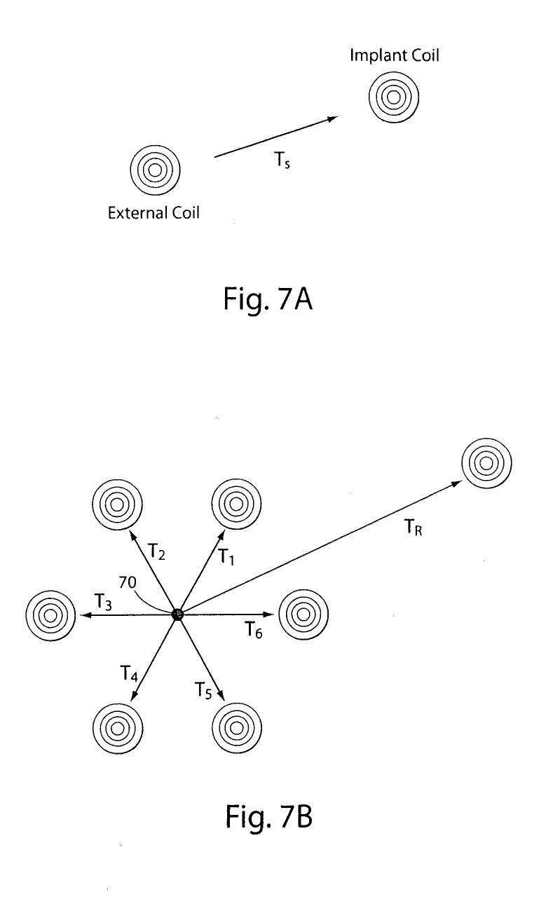

FIGS. 7A and 7B, in connection with the following description, describe a method of computing position and orientation information indicative of how closely a port locator is aligned with an implanted port, in accordance with some embodiments of the present invention. In particular, the method includes determining the transformation between a known location on the port locator and the port that best explains a plurality of measurements obtained from an implanted coil. FIG. 7A illustrates a basic sensor system having an external coil (i.e., a port locator coil) in a generally unknown proximity to an implanted coil (i.e., a port beacon coil). For the sake of clarity, the various other components of the port locator and port beacon (e.g., any of various components described in the foregoing) have been omitted.

The behavior of the system in FIG. 7A may be characterized by a model that predicts the signal that will be measured as a function of the coordinate transformation (transform), Tₛ, that relates the two coils. That is, the transformation that, if applied to the external coil would position the external coil in the same location and orientation as the implanted coil. The predicted model may be expressed as follows:
x=f(Tₛ) (1),

where x is the predicted signal (e.g., the predicted echo signal generated by an implant coil whose spatial relationship with the external coil is described by transform Tₛ). FIG. 7B illustrates a port locator system including six port locator coils 705, each of which is offset from an origin 70 of a port locator coordinate system by a known transform, Tᵢ where i takes on values from 1 to the number of port locator coils. It should be appreciated that the configuration of the coils in FIG. 7B is exemplary and schematic, and that any configuration may be used as long as the spatial relationship between the port locator coils is known. If the relative transform that relates the origin of the port locator and the implant coil is given by T_{R}, then the transform that relates port locator coil i to the implant coil will be given by Tᵢ⁻¹T_{R}, and the measured echo signal when using coil i will be predicted by:
xᵢ=f(Tᵢ⁻¹T_{R}) (2).

Given corresponding measured signals Yᵢ actually obtained through operation of the system, and substituting the actual measure signals Yᵢ for the predicted signal xᵢ, the unknown transformation T_{R} may be estimated by minimizing the following expression:

$$E = {\sum }_{i} {[ {Y}_{i} - f ( {T}_{i}^{- 1} {T}_{R} ) ]}^{2} . \qquad ( 3 )$$

That is, the parameters of the unknown relative transform T_{R} may be estimated by choosing the parameters to minimize the total error, E. In general, this is a problem of nonlinear optimization that may be solved with standard methods such as gradient decent, or may be solved using any suitable optimization method. It should be appreciated that a program to be executed on a processor of the port locator (e.g., processor 550′) can be written to solve the optimization problem discussed above. Thus, the port locator can both determine position and orientation information with respective to the relationship between the port locator and the implanted port. The processor may then use this information to provide indication to the feedback mechanism as to how well the port locator is aligned with implanted port.

It should be appreciated that the above method for determining position and orientation information from measurements detected at a plurality of coils is merely exemplary, and other methods may be used, as the aspects of the invention are not limited in this respect. It should be appreciated that additional implant coils may be used to obtain additional information about the location of the implant coil. Any number of and/or configuration of port locator coils and implant coils may be used, as the aspects of the invention are not limited in this respect.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed function. The one or more controller can be implemented in numerous ways, such as with dedicated hardware, or with general purpose hardware (e.g., one or more processor) that is programmed using microcode or software to perform the functions recited above.

It should be appreciated that the various methods outlined herein may be coded as software that is executable on one or more processors that employ any one of a variety of operating systems or platforms. Additionally, such software may be written using any of a number of suitable programming languages and/or conventional programming or scripting tools, and also may be compiled as executable machine language code.

In this respect, it should be appreciated that one embodiment of the invention is directed to a computer readable medium encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

It should be understood that the term “program” is used herein in a generic sense to refer to any type of computer code or set of instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. The invention is capable of other embodiments and of being practiced or of being carried out in various ways. In particular, the various concepts related to variable radiation energy and variable radiation intensity may be used in any way, either alone or in any combination, as the aspects of the invention are not limited to the specific combinations described herein. Accordingly, the foregoing description and drawings are by way of example only.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

What is claimed is:

1. A port beacon adapted to be implanted with a subcutaneous port to facilitate locating the implanted port, the port beacon comprising:
at least one coil configured to obtain first energy from an externally provided excitation signal and to transmit an echo signal in response to the excitation signal; and
a transmit and receive circuit coupled to the at least one coil, the transmit and receive circuit configured to receive the first energy obtained by the at least one coil and provide the at least one coil with second energy derived, at least in part, from the first energy to cause the at least one coil to transmit the echo signal.

2. The port beacon of claim 1, wherein the transmit and receive circuit includes a harmonic generator coupled to the at least one coil, and wherein the excitation signal is provided at a first frequency and the harmonic generator converts the first energy into a drive signal having at least one harmonic of the first frequency such that the echo signal is provided at the at least one harmonic of the first frequency.

3. The port beacon of claim 1, wherein the transmit and receive circuit includes a capacitor capable of being charged with at least some of the first energy.

4. The port beacon of claim 2, wherein the transmit and receive circuit includes a drive signal generator coupled with the capacitor and the at least one coil, the drive signal generator receiving current from the capacitor, when discharging, and generating a drive signal to energize the at least one coil to transmit the echo signal.

5. The port beacon of claim 4, wherein the excitation signal includes at least one electromagnetic pulse, and wherein the transmit and receive circuit is capable of operating in a receive mode and a transmit mode, wherein, during the receive mode, the capacitor is charged using at least some of the first energy obtained from the at least one electromagnetic pulse and, during the transmit mode, the capacitor is discharged to provide the current to the drive signal generator to cause the at least one coil to transmit the echo signal.

6. The port beacon of claim 5, wherein the drive signal generator is an oscillator configure to generate the drive signal at a desired frequency.

7. A port locator for facilitating location of an port implanted subcutaneously, the port locator comprising:
at least one coil responsive to an electromagnetic beacon signal generated at the port, the at least one coil configured to generate a locator signal in response to the electromagnetic beacon signal; and
a controller configure to receive the locator signal and generate at least one proximity measurement indicative of a location of the port based, at least in part, on the locator signal.

8. The port locator of claim 7, wherein the controller is configured to generate at least one feedback signal indicative of the location of the port based, at least in part, on the at least one proximity measurement.

9. The port locator of claim 8, wherein the controller is configured to generate at least one feedback signal indicative of how closely aligned the port locator is aligned with the port.

10. The port locator of claim 7, wherein the at least one feedback signal includes at least one visual feedback signal.

11. The port locator of claim 8, wherein the at least one feedback signal includes at least one audio feedback signal.

12. The port locator of claim 11, wherein the at least one proximity measurement varies in magnitude as a function of a magnitude of the electromagnetic beacon signal, and wherein the at least one audio feedback signal varies in frequency as a function of the magnitude of the proximity measurement.

13. The port locator of claim 11, wherein the at least one proximity measurement varies in magnitude as a function of a magnitude of the electromagnetic beacon signal, and wherein the at least one audio feedback signal varies in volume as a function of the magnitude of the proximity measurement.

14. The port locator of claim 7, wherein the at least one coil includes a plurality of coils to generate a respective plurality of locator signals, and wherein the controller is configured to receive the plurality of locator signals and generate the at least one proximity measurement indicative of the location of the port based, at least in part, on the plurality of locator signals.

15. The port locator of claim 14, wherein the at least one proximity measurement includes position information and orientation information indicative of a position and orientation of the implanted port, respectively.

16. The port locator of claim 14, wherein the port locator includes a bore adapted to accept a needle to access the subcutaneous port, and wherein the at least one proximity measurement includes position information and orientation information indicative of a transform between a center of the implanted port and the bore.

17. A device for locating a subcutaneously implanted port, the device comprising:
a port beacon adapted to be implanted proximate the subcutaneously implanted port, the port beacon configured to generate a beacon signal; and
a port locator comprising at least one first coil responsive to the beacon signal, the at least one first coil configured to generate a locator signal in response to the beacon signal, the port locator further comprising a controller to receive the locator signal and generate at least one proximity measurement indicative of a location of the port beacon based, at least in part, on the locator signal.

18. The device of claim 17, wherein the at least one first coil is adapted to transmit an excitation signal and the port beacon is configured to detect the excitation signal and generate the beacon signal in response to the excitation signal.

19. The device of claim 18, wherein the port beacon includes at least one induction coil adapted to detect the excitation signal and to generate the beacon signal.

20. The device of claim 18, wherein the at least one first coil includes a plurality of first coils adapted to generate a respective plurality of locator signals in response to the beacon signal, and wherein the controller is configured to receive the plurality of locator signals and to generate the at least one proximity measurement based, at least in part, on the plurality of locator signals.

21. The device of claim 20, wherein the at least one proximity measurement includes position information and orientation information indicative of the location of the port beacon with respect to the port locator.

22. The device of claim 21, wherein the position information and orientation information is indicative of a spatial transform between a predetermined location on the port locator and the port beacon.

23. The device of claim 22, wherein the port locator includes a bore adapted to accept a needle for accessing the port, and wherein port beacon is arranged in a known location with respect to an opening in the port, and wherein the position information and orientation information is indicative of a spatial transform between the bore and the opening in the port.

24. The device of claim 20, wherein the controller is configured select any of the plurality of coils to transmit the excitation signal, and to select any of the plurality of coils at which to receive the respective locator signal.

25. The device of claim 24, wherein the controller is configured energize coils of the plurality of coils to transmit excitation signals and select coils of the plurality of coils to receive locator signals according to a desired sequence to obtain the plurality of locator signals.

26. The device of claim 21, wherein the controller is configured to generate at least one feedback signal that indicates to a user of the device how closely the port locator is aligned with the port beacon.

27. The device of claim 26, wherein the at least one feedback signal includes at least one audio feedback signal.

28. The device of claim 26, wherein the at least one feedback signal includes at least one visual feedback signal.

29. A method of locating a port beacon implanted subcutaneously in a patient proximate an implanted port, the method comprising:
generating, at the port beacon, an electromagnetic beacon signal;
detecting the electromagnetic beacon signal via at least one first coil provided external to the patient; and
determining at least one proximity measurement indicative of a location of the port beacon based, at least in part, on the detected electromagnetic beacon signal.

30. The method of claim 29, wherein generating the electromagnetic beacon signal includes generating the electromagnetic beacon signal via at least one second coil.

31. The method of claim 30, further comprising generating an excitation signal via the at least one first coil.

32. The method of claim 31, further comprising detecting the excitation signal via the at least one second coil, wherein generating the electromagnetic beacon signal includes generating the electromagnetic beacon signal in response to detecting the excitation signal.

33. The method of claim 32, wherein generating the electromagnetic beacon signal includes using energy obtained from the excitation signal to drive the at least one second coil to generate the electromagnetic beacon signal.

34. The method of claim 32, wherein generating the excitation signal includes generating a plurality of excitation signals from a respective plurality of first coils, and wherein detecting the electromagnetic beacon signal includes detecting, via the plurality of first coils, a plurality of electromagnetic beacon signals generated in response to respective ones of the plurality of excitation signals.

35. The method of claim 34, wherein determining the at least one proximity measurement includes determining position information and orientation information indicative of a position and orientation of the port beacon based, at least in part, on the plurality of detected electromagnetic beacon signals.

36. The method of claim 35, further comprising generating at least one feedback signal indicative of the location of the port based on the position information and the orientation information.
